(19) United States
(12) Patent Application Publication
(10) Pub. No.: US 2003/0039706 A1
(43) Pub. Date: Feb. 27, 2003

(54) Sialagogue, and food composition and oral composition containing thereof

(76) Inventors: Kazuo Hirose, Osaka (JP); Shinichi Matsumura, Osaka (JP); Tohru Nakasugi, Osaka (JP)

Correspondence Address: S. Alex Liao, Suite 905, 12 South First St., San Jose, CA, 95113, US

(21) Appl. No.: 10/097,720

(22) Filed: Mar. 13, 2002

(30) Foreign Application Priority Data
Mar. 13, 2001 (JP) 2001-70258

(51) Int. Cl. A61K 35/78

(52) U.S. Cl. 424/725

(57) ABSTRACT

This invention is to provide sialagogue wherein one or more kind of plant and/or extract thereof selected from the group of the plant belonging to Capparidaceae and the plant belonging to Umbelliferae are contained, and food composition and oral composition containing thereof. The sialagogue, and the food composition and the oral composition containing thereof, which cure dryness of a mouth by promoting salivary secretion having more than fixed quantity consecutively for a long time, and which can prevent generating dental caries by raising the self-cleansing action in the mouth.

### FIELD OF THE INVENTION

This invention relates to sialagogue, and food composition and oral composition containing thereof, and the object thereof is to provide the sialagogue, and the food composition and the oral composition containing thereof, which cure dryness of a mouth by promoting salivary secretion having more than fixed quantity consecutively for a long time, and which can prevent generating dental caries by raising the self-cleansing action in the mouth.

### PRIORART

The saliva secreted from the salivary gland has a lot of functions: digestion, assertive action for mastication, solvent action, smooth action for chatting and vocalizing, cleaning operation, pH buffering action and so on, whereas, it is well-known that the secretion quantity of the saliva is declined because of an emotional disorder, a neurosis, an internal organ derangement, encephalitis, a neoplasm, a cerebral vascular accident, hypertension, all kinds of diseases such as the diabetes, adverse reaction of medicine, radiotherapy and so on. There are many people complaining the dryness in the mouth, in particular, the aged people, since the secretion quantity of the saliva is decreased by aging, a plurality of chronic disease or their cure medicine.

Dryness in a mouth with decreasing secretion quantity of the saliva will cause indisposition, the occurrence of the halitosis and so on, and further progressive of the disease will cause periodontal disease, infection disease and so on. Thus, it is desired to increase the secretion quantity of the saliva of the patient having the symptom as the above by some means.

It is thought that the dental caries occur since the bacteria inhabiting the surface of the tooth decompose sugar and the like so that the pH in the mouth is declined and dental enamel of the surface of the tooth is dissolved. Meanwhile, in usual case, the occurrence of the dental caries can be prevented because the secretion of the saliva is promoted by having a meal so that the acid in the mouth is neutralized by the buffering action of the saliva.

When the secretion of the saliva decreases for some reasons, for example, an internal organ derangement, hypertension, all kinds of diseases such as the diabetes, adverse reaction of medicine, and radiotherapy, the dental caries will be easy to generate by declining the buffering action of the saliva.

In order to increase the secretion quantity of the saliva, whereas there are some methods: increasing a number of mastication; irritating; and utilizing secretion promotion action of the saliva by gustatory substance, these methods are transient during mastication and they are impossible to secrete consecutively for more than certain time. It is hard to masticate for the aged people, in particular so it is difficult to apply such method itself.

Japan patent publication H10-182392 discloses sialagogue containing seed powder of Cola trees (Cola acuminata and Cola nitida A. Chev) and organic acid. Also, Japan patent publication H11-71253 discloses sialagogue containing arborvitae fruit and organic acid.

However, since those sialagogue contains organic acid as essential component even though secretion of saliva by working organic acid is promoted, it will be cause of dental caries because of declining pH in a mouth and there is possibility of danger to damage a tooth by strong irritating of acid.

This inventor has continued the research and found that saliva with more than certain quantity can be secreted consecutively for a long time by using the plant belonging to Cappaidaceae and the plant belonging to Umbelliferae as essential components, and has invented the present invention.

### SUMMERY OF THE INVENTION

The invention concerning claim 1 relates sialagogue wherein one or more kind of plant and/or extract thereof selected from the group of the plant belonging to Capparidaceae and the plant belonging to Umbelliferae are contained.

The invention concerning claim 2 relates sialagogue wherein one or more kind of plant and/or extract thereof selected from the group of the plant belonging to Capparidaceae and Capparis, and the plant belonging to Umbelliferae and Centella are contained.

The invention conceming claim 3 relates sialagogue wherein one or more kind of plant and/or extract thereof selected from the group of Capparis pterocarpa Chun, Capparis masaikai Levl. and Capparis asiatica (L.) Urban are contained.

The invention concerning claim 4 relates sialagogue claimed in claim 1, wherein organic acid is contained.

The invention concerning claim 5 relates sialagogue claimed in claim 2, wherein organic acid is contained.

The invention concerning claim 6 relates sialagogue claimed in claim 3, wherein organic acid is contained.

The invention concerning claim 7 relates sialagogue claimed in claim 1, wherein organic acid and sugar alcohol are contained.

The invention concerning claim 8 relates sialagogue claimed in claim 2, wherein organic acid and sugar alcohol are contained.

The invention concerning claim 9 relates sialagogue claimed in claim 3, wherein organic acid and sugar alcohol are contained.

The invention concerning claim 10 relates sialagogue claimed in claim 1, wherein organic acid, sugar alcohol and at least one selected from the group consisting of peppermint oil, cinnamon oil, menthol and cinnamic aldehyde are contained.

The invention concerning claim 11 relates sialagogue claimed in claim 2, wherein organic acid, sugar alcohol and atleast one selected from the group consisting of peppermint oil, cinnamon oil, menthol and cinnamic aldehyde are contained.

The invention concerning claim 12 relates sialagogue claimed in claim 3, wherein organic acid, sugar alcohol and at least one selected from the group consisting of peppermint oil, cinnamon oil, menthol and cinnamic aldehyde are contained.

The invention concerning claim 13 relates sialagogue claimed in claim 1, wherein fermentable sugar is contained.

The invention concerning claim 14 relates sialagogue claimed in claim 2, wherein fermentable sugar is contained.

The invention concerning claim 15 relates sialagogue claimed in claim 3, wherein fermentable sugar is contained.

The invention concerning claim 16 relates sialagogue claimed in claim 1, wherein fermentable sugar and sugar alcohol are contained.

The invention concerning claim 17 relates sialagogue claimed in claim 2, wherein fermentable sugar and sugar alcohol are contained.

The invention concerning claim 18 relates sialagogue claimed in claim 3, containing fermentable sugar and sugar alcohol.

The invention concerning claim 19 relates sialagogue claimed in claim 1, wherein fermentable sugar, sugar alcohol and at least one selected from the group consisting of peppermint oil, cinnamon oil, menthol and cinnamic aldehyde are contained.

The invention concerning claim 20 relates sialagogue claimed in claim 2, wherein fermentable sugar, sugar alcohol and at least one selected from the group consisting of peppermint oil, cinnamon oil, menthol and cinnamic aldehyde are contained.

The invention concerning claim 21 relates sialagogue claimed in claim 3, wherein fermentable sugar, sugar alcohol and at least one selected from the group consisting of peppermint oil, cinnamon oil, menthol and cinnamic aldehyde are contained.

The invention concerning claim 22 relates sialagogue claimed in claim 1, wherein one or more kind of which selected from the group of organic acid, fermentable sugar, sugar alcohol, peppermint oil, cinnamon oil, menthol and cinnamic aldehyde are contained.

The invention concerning claim 23 relates sialagogue claimed in claim 2, wherein one or more kind of which selected from the group of organic acid, fermentable sugar, sugar alcohol, peppermint oil, cinnamon oil, menthol and cinnamic aldehyde are contained.

The invention concerning claim 24 relates sialagogue claimed in claim 3, wherein one or more kind of which selected from the group of organic acid, fermentable sugar, sugar alcohol, peppermint oil, cinnamon oil, menthol and cinnamic aldehyde are contained.

The invention concerning claim 25 relates food composition wherein sialagogue claimed in claim 1 is compounded.

The invention concerning claim 26 relates food composition wherein sialagogue claimed in claim 2 is compounded.

The invention concerning claim 27 relates food composition wherein sialagogue claimed in claim 3 is compounded.

The invention concerning claim 28 relates food composition wherein sialagogue claimed in claim 4 is compounded.

The invention concerning claim 29 relates food composition wherein sialagogue claimed in claim 5 is compounded.

The invention concerning claim 30 relates food composition wherein sialagogue claimed in claim 6 is compounded.

The invention concerning claim 31 relates food composition wherein sialagogue claimed in claim 7 is compounded.

The invention concerning claim 32 relates food composition wherein sialagogue claimed in claim 8 is compounded.

The invention concerning claim 33 relates food composition wherein sialagogue claimed in claim 9 is compounded.

The invention concerning claim 34 relates food composition wherein sialagogue claimed in claim 10 is compounded.

The invention concerning claim 35 relates food composition wherein sialagogue claimed in claim 1 is compounded.

The invention concerning claim 36 relates food composition wherein sialagogue claimed in claim 12 is compounded.

The invention concerning claim 37 relates food composition wherein sialagogue claimed in claim 13 is compounded.

The invention concerning claim 38 relates food composition wherein sialagogue claimed in claim 14 is compounded.

The invention concerning claim 39 relates food composition wherein sialagogue claimed in claim 15 is compounded.

The invention concerning claim 40 relates food composition wherein sialagogue claimed in claim 16 is compounded.

The invention concerning claim 41 relates food composition wherein sialagogue claimed in claim 17 is compounded.

The invention concerning claim 42 relates food composition wherein sialagogue claimed in claim 18 is compounded.

The invention concerning claim 43 relates food composition wherein sialagogue claimed in claim 19 is compounded.

The invention concerning claim 44 relates food composition wherein sialagogue claimed in claim 20 is compounded.

The invention concerning claim 45 relates food composition wherein sialagogue claimed in claim 21 is compounded.

The invention concerning claim 46 relates food composition wherein sialagogue claimed in claim 22 is compounded.

The invention concerning claim 47 relates food composition wherein sialagogue claimed in claim 23 is compounded.

The invention concerning claim 48 relates food composition wherein sialagogue claimed in claim 24 is compounded.

The invention concerning claim 49 relates oral composition wherein sialagogue claimed in claim 1 is compounded.

The invention concerning claim 50 relates oral composition wherein sialagogue claimed in claim 2 is compounded.

The invention concerning claim 51 relates oral composition wherein sialagogue claimed in claim 3 is compounded.

The invention concerning claim 52 relates oral composition wherein sialagogue claimed in claim 4 is compounded.

The invention concerning claim 53 relates oral composition wherein sialagogue claimed in claim 5 is compounded.

The invention concerning claim 54 relates oral composition wherein sialagogue claimed in claim 6 is compounded.

The invention concerning claim 55 relates oral composition wherein sialagogue claimed in claim 7 is compounded.

The invention concerning claim 56 relates oral composition wherein sialagogue claimed in claim 8 is compounded.

The invention concerning claim 57 relates oral composition wherein sialagogue claimed in claim 9 is compounded.

The invention concerning claim 58 relates oral composition wherein sialagogue claimed in claim 10 is compounded.

The invention concerning claim 59 relates oral composition wherein sialagogue claimed in claim 11 is compounded.

The invention concerning claim 60 relates oral composition wherein sialagogue claimed in claim 12 is compounded.

The invention concerning claim 61 relates oral composition wherein sialagogue claimed in claim 13 is compounded.

The invention concerning claim 62 relates oral composition wherein sialagogue claimed in claim 14 is compounded.

The invention concerning claim 63 relates oral composition wherein sialagogue claimed in claim 15 is compounded.

The invention concerning claim 64 relates oral composition wherein sialagogue claimed in claim 16 is compounded.

The invention concerning claim 65 relates oral composition wherein sialagogue claimed in claim 17 is compounded.

The invention concerning claim 66 relates oral composition wherein sialagogue claimed in claim 18 is compounded.

The invention concerning claim 67 relates oral composition wherein sialagogue claimed in claim 19 is compounded.

The invention concerning claim 68 relates oral composition wherein sialagogue claimed in claim 20 is compounded.

The invention concerning claim 69 relates oral composition wherein sialagogue claimed in claim 21 is compounded.

The invention concerning claim 70 relates oral composition wherein sialagogue claimed in claim 22 is compounded.

The invention concerning claim 71 relates oral composition wherein sialagogue claimed in claim 23 is compounded.

The invention concerning claim 72 relates oral composition wherein sialagogue claimed in claim 24 is compounded.

### DETAILED DESCRIPTION OF THE INVENTION

Hereinafter, sialagogue, and food composition and oral composition containing thereof, which are related to the present invention is described.

The sialagogue concerning the present invention contains one or more kind of plant selected from the group of the plant belonging to Capparidaceae and the plant belonging to Umbelliferae.

As for the plant belonging to Capparidaceae, it is not limited, in particular, but a plant belonging to Capparidaceae and Capparis can be exemplified, and concretely, Capparis pterocarpa Chun and Capparis masaikai Levl. can be exemplified.

As for the plant belonging to Umbelliferae, it is also not limited, in particular, but a plant belonging to Umbelliferae and Centella can be exemplified, and concretely, Centella asiatica (L.) Urban can be exemplified.

Capparis pterocarpa Chun is the plant belonging to Capparidaceae and Capparis, is distributed throughout Guangdong, Guangxi, Yunnan and so on in China. It is a crane bush and becomes more than 3 m height. The dehydrated seed is used as the herb medicine.

In case of the present invention, each part of the leaf part, the trunk part, the root part, the seed and so on can be used independently or as mixture appropriately. Incidentally, using a seed part in the present invention is preferable.

Capparis masaikai Levl. is the plant belonging to Capparidaceae and Capparis, is distributed throughout Guangdong, Guangxi, Yunnan, Guizhou and so on in China. The dehydrated seed is used as the herb medicine. Incidentally, the plant is caged “babinlow” or “mabinlow” in Japanese.

In case of the present invention, each part of the leaf part, the trunk part, the root part, the seed and so on can be used independently or as mixture appropriately. Incidentally, using a seed part in the present invention is preferable.

Centella asiatica (L.) Urban is the plant belonging to a plant belonging to Umbelliferae and Centella, is distributed throughout Southern part of Kanto area, Shikoku, Kyusyu and Okinawa in Japan, Korean peninsula, Taiwan, China and so on. The dehydrated grass is used as the herb medicine and has detoxification effect hemostasis effect, diuretic effect and the like.

In case of the present invention, each part of the whole grass or the leaf part, the trunk part, the seed and so on can be used independently or as mixture appropriately. Incidentally, using a seed part in the present invention is preferable.

In the present invention, the above-mentioned plants or the dehydrated fragment thereof can be used just as it is but the extractive thereof can be also used. The extractive solvent is not limited in particular, but following can be exemplified: acid such as hydrochloric acid and acetic or alkali such as sodium hydroxide solution and sodium carbonate; in addition, water, acetone; acetonitrile; benzene; chloroform; cyclohexane; diethyl ether, diisopropyl ether, N, N-dimethylacetamide; N, N-dimethylformamide; 1,4-dioxane; ethanol; ethyl acetate; ethylene glycol dimethyl ether, heptane; hexane; methanol; N-methyl-2-pyrrolidinone; n-pentane; 2-propanol; pyridine; tetrahydrofuran; toluene; xylene; chlorobenzene; dichloromethane; ethylene glycol; formamide; 2-methoxyethanol; anisole; 2-butanol; methyl formate; isobutyl acetate; isopropyl acetate; 3-methyl-1-butanol; methyl ethyl ketone; methyl isobutyl ketone; 2-methyl-l-butanol; pentane; 1-pentanol; 1-propanol; 2-propanol; petroleum ether, ligroin; isooctane and so on. Furthermore, mixed solvent mixing more than one kind of solvent may be used. In the present invention in particular, water and ethanol are preferably used.

Extractive method is not limited in particular, but following methods can be exemplified: a method wherein extractive solvent is evaporated with depression after the dehydrated fragment of said plant is extracted with dipping into 1 to 100 weight times of extractive solvent thereof for 1 to 30 days; a method wherein the dehydrated fragment of said plant is extracted with reflux for about 1 to 5 hours using extractive solvent heard to reflux temperature.

As for the sialagogue concerning the present invention, the other ingredients, which have the secretion promotion effect of the saliva, can be used with compounding appropriately. As the ingredients having the secretion promotion effect of the saliva, organic acid, sugar alcohol, flavor and so on can be exemplified.

As organic acid, citric acid, isocitric acid, malic acid, acetic acid, oxalic acid, malonic acid, succinic acid, glutaric acid, maleic acid, fumaric acid, aconitic acid, lactic acid, tartaric acid, pyruvic acid, ascorbic acid and so on can be exemplified, and using citric acid, malic acid, acetic acid and lactic acid is preferable.

The organic acid can promote the secretion of the saliva even it is independent; however, when it is used with said plant or extractive thereof, it can promote the secretion of the saliva more remarkably comparing to the case using them independently. If the secretion of the saliva is promoted, the pH decline in the mouth can be prevented and the occurrence of dental caries can be prevented.

When the organic acid is compounded, the amount is not limited in particular, however, if the organic acid is compounded too much, there maybe danger of occurrence of dental caries because of the pH decline in the mouth; therefore, the amount of organic acid is 0.01 to 100 part by weight to 1 part by weight of said plant or extractive thereof, and preferably 0.1 to 1 part by weight.

As sugar alcohol, xylitol, maltitol, sorbitol, deoxidization paratinose, erythritol, lactitol and so on can be exemplified Xylitol, maltitol and sorbitol can be preferably used in the present inventon.

The sugar alcohol can promote the secretion of the saliva even it is independent; however, when it is used with said plant or extractive thereof, it can promote the secretion of the saliva more remarkably comparing to the case using them independently. If the secretion of the saliva is promoted, the pH decline in the mouth can be prevented and the occurrence of dental caries can be prevented.

The amount of sugar alcohol is not limited in particular; however, if the sugar alcohol is compounded too much, there may be danger of occurrence of dental caries because of the pH decline in the mouth; therefore, the amount of sugar alcohol is 0.01 to 100 part by weight to 1 part by weight of said plant or extractive thereof, and preferably 0.1 to 1 part by weight.

As flavor, peppermint oil, cinnamon oil, menthol which is the main ingredient of peppermint oil, or cinnamic aldehyde which is the main ingredient of cinnamon oil is preferably used. When said flavor is used, it can promote the secretion of the saliva more remarkably comparing to the case using the other flavor.

The flavor can promote the secretion of the saliva even it is independent; however, when it is used with said plant or extractive thereof, it can promote the secretion of the saliva more remarkably comparing to the case using them independently. If the secretion of the saliva is promoted, the pH decline in the mouth can be prevented and the occurrence of dental caries can be prevented.

The amount of flavor is not limited in particular, however, if the flavor is compounded too much, there maybe danger of occurrence of dental caries because of the pH decline in the mouth; therefore, the amount of flavor is 0.01 to 100 part by weight to 1 part by weight of said plant or extractive thereof, and preferably 0. 1 to 1 part by weight.

Moreover, the sialagogue concerning the present invention can be compounded fermentable sugar to improve gustation and so on. Since the sialagogue concerning the present invention promotes the secretion of the saliva, the pH decline in the mouth can be prevented and the occurrence of dental caries can be prevented even if the fermentable sugar is compounded. As the fermentable sugar, glucose, sucrose, fructose and so on can be illustrated.

The amount of fermentable sugar is not limited in particular, however, if the fermentable sugar is compounded too much there maybe danger of occurrence of dental caries because of the pH decline in the mouth; therefore, the amount of fermentable sugar is 0.01 to 100 part by weight to 1 part by weight of said plant or extractive thereof, and preferably 0.1 to 1 part by weight.

In addition, when said organic acid, sugar alcohol, flavor, and fermentable sugar are compounded, combination thereof is not limited in particular, but following combination, when the organic acid is compounded with said plant or extractive thereof, can be exemplified: a combination of said plant or extractive thereof and the organic acid; a combination of said plant or extractive thereof, the organic acid and sugar alcohol; a combination of said plant or extractive thereof, the organic acid, sugar alcohol and flavor; and so on.

Further, following combination, when the fermentable sugar is compounded with said plant or extractive thereof, can be exemplified: a combination of said plant or extractive thereof and the fermentable sugar, a combination of said plant or extractive thereof, the fermentable sugar and sugar alcohol; a combination of said plant or extractive thereof, the fermentable sugar, sugar alcohol and flavor; and so on.

Whereas the sialagogue concerning the present invention can be use as it is or with diluting with water, it may use with compounding into oral composition and/or food composition.

In this case, the compounding amount of sialagogue is not limited in particular, however, it will be 0.01 to 50 weight %, preferably 0.1 to 10 weight %, and more preferably 1 to 5 weight %, in whole components.

When the sialagogue concerning the present invention is used with compounding into oral composition, composition compounded in usual oral composition may be compounded as appropriate.

The composition to be compounded is not limited in particular, but viscosity, active agent, adhesive, abrasive, sweetener, sterilizer, coloring agent, flavor, all kinds of active ingredient and so on can be exemplified. Concretely, following can be shown as example. As viscosity: glycerine, sorbit, polyethylene glycol, xylit, Mannit and so on; as active agent: sodium lauryl sulfate,α-sodium oleate, sucrose fatty acid ester, alkylolamid, polyoxyethylene hardened castor oil, polyoxyethylene sorbitan monooleate and so on; as adhesive, carboxymethyl cellulose, ethyl hydroxyethyl cellulose, carragheenan, xanthan gum, Cyamoposis Gum, gelatin and so on; as abrasive, silica gel aluminosilicate, dibasic calcium phosphate, calcium carbonate, alumina, magnesium Carbonate and so on; as sweetener, saccharin sodium, stevia so on; as sterilizer, p-hydroxybenzoic esters, sodium benzoate and so on; as coloring agent, blue No.1, yellow No.4 and so on; as flavor, menthol, limonene, anethole and so on; as all kinds of active ingredient, fluoride, water soluble phosphate, lysozyme chloride, glycyrrhizinate, tranexanic acid, strontium chloride, ligusticum extract; caiyophyllus extract, scutellariae radix extract benzethonium chloride and so on.

The physical state of the oral composition is not limited in particular, but the one which uses for mouth to promote secretion of the saliva directly, such as toothpaste, mouth rinse, ointment for mouth, tablet for gargle, lozenge, mastication tablet; sprayer for mouth, can be exemplified.

When the sialagogue concerning the present invention is used with compounding into food composition, composition compounded in usual oral composition may be compounded as appropriate.

The composition to be compounded is not limited in particular, but following can be exemplified: milk fat, animal oil such as beef tallow, olive oil, cacao oil, sesame oil, lincoln bean oil, corn oil, vegetable oil such ascottonseed oil, saccharose, fructose, glucose, paratinose, fractsaccharide, dextrin, aspartame, sweetener such as sugar alcohol, benzoic acid, sorbic acid, p-hydroxybenzoic acid, preservative such as propionic acid, orthophenyl phenol, fungicide such as thiabendazde, ascorbic acid, ethylene-diamine-tetraacetic acid disodium calcium, isopropyl citrate, starch or antioxidant such as dibutyl hydroxytoluene, thickener, gelatinizer starch adhesive, dietary fiber, flavor enhancer, food color, vitamin, saline, vinegar, soy sauce, spice, ferment and so on.

The physical state of the food composition is not limited in particular however, candy, beverage, chewing gum, tablet cookie, cake, frozen sweets, jelly, pasta and so on can be exemplified.

### EXAMPLE

Hereinafter, the present invention is described based on examples but it does not mean to limit the present invention.

1. Preparation of Sample

11000 ml of 50% ethanol was added into 100 g of dehydrate fragment of seed of Capparis pterocarpa Chun and it mixed for three hours, and then the solvent was evaporated with depression so that 10 g of Capparis pterocarpa Chun extractive was obtained as Sample 1.

100 g of dehydrate fragment of seed of Capparis masaikai Levl. was as Sample 2.

1000 ml of 50% ethanol was added into 100 g of dehydrate fragment of seed of Centella asiatica (L.) Urban and it mixed for three hours, and then the solvent was evaporated with depression so that 10 g of Centella asiatica (L.) Urban extractive was obtained as Sample 3.

Next, according to the following composition, each samples (0.2 g of tablet per one piece) of Examples 1 to 3 were prepared by compounding 5.0 part by weight of samples 1 to 3, respectively. Also, samples (0.2 g of tablet per one piece) of comparative example 1 were prepared without compounding any samples 1 to 3.

Tablet

| | | |
|---|---|---|
| Glucose: | 97.5 | part by weight |
| Sucrose esters: | 2.5 | part by weight |
| Sample in examples or comparative example: | 5.0 | part by weight |

2. Test: Measurement of Amount of Salivary Secretion

First, amount of the salivary secretion of testee during taking a rest was measured. The way of measuring amount of the salivary secretion is as follows: after saliva in mouth was wiped off with gauze, a cotton roll (32 mm length, 12 mm diameter in column) was put into mouth for one minute, and then amount of saliva absorbed into the cotton roll was measured.

Next, each of the samples of Examples 1 to 3, prepared as above is administrated with one piece per sample to twenty testees The amount of salivary secretion at 10 minutes, 20 minutes and 30 minutes after the administration was also measured as same way as described above.

On the next day when each sample of the Examples were administrated and amount of salivary secretion was measured, amount of the salivary secretion during taking a rest was measured as the same way as described above and amount of the salivary secretion when sample of the comparative example 1 was administrated was measured.

The average of amount of salivary secretion is shown in Table 1.

TABLE 1

| | | | 10 | 20 | 30 |
|---|---|---|---|---|---|
| | Amount of | Before | minutes | minutes | minutes |
| | combination | administration | later | later | later |
| Example | 5% | 0.44 g | 0.70 g | 0.67 g | 0.63 g |
| 1 | 0% | 0.43 g | 0.51 g | 0.45 g | 0.42 g |
| Example | 5% | 0.43 g | 0.65 g | 0.59 g | 0.56 g |
| 2 | 0% | 0.49 g | 0.53 g | 0.50 g | 0.49 g |
| Example | 5% | 0.42 g | 0.58 g | 0.55 g | 0.54 g |
| 3 | 0% | 0.41 g | 0.50 g | 0.42 g | 0.41 g |

As the result in Table 1, regarding the sample of the Example, it is found that amount of salivary secretion is a lot compared with the case in a rest even it is passed 30 minutes after administration. On the other hand, in case of sample of the comparative example, it is found that is almost same amount as the amount of salivary secretion in the case of a rest after passing for 20 minutes whereas amount of salivary secretion increased immediately after administration. Thus, the sialagogue concerning the present invention can promote salivary secretion having more than fixed quantity consecutively for a long time.

3. Test 2: Influence on pH Decline in Mouth

(1) Preparation of Sample

Control was prepared by compounding 95 weight % of glucose with 5 weight % of citric acid

Example 4 was prepared by compounding 85 weight % of glucose with 5 weight % of citric acid and 10 weight % of said sample 2.

Example 5 was prepared by compounding 80 weight % of glucose with 5 weight % of citric acid, 10 weight % of said sample 2 and 5 weight % of xylitol.

Example 6 was prepared by compounding 79 weight % of glucose with 5 weight % of citric acid, 10 weight % of said sample 2,5 weight % of xylitol and 1 weight % of peppermint oil.

Example 7 was prepared by compounding 79.2 weight % of glucose with 5 weight % of citric acid, 10 weight % of said sample 2, 5 weight % of xylitol and 0.8 weight % of cinnamaldehyde.

(2) Test Method

First, the saliva of (five) testee immediately before administrating said sample was collected, was diluted with appropriate amount of water (30 ml) and then pH value of the solution was measured. It is set as the pH value on normal. Next, after each sample (250 mg) was administrated, the pH value of the saliva secreted in three minutes was measured as same as described above, and then it was compared with the pH value of the saliva before administrating sample. the average of the five testee is shown in Table 2. Incidentally, in order to check that the change of the pH value was not merely neutralization effect of the additional ingredient, the pH value of 250 mg/30 mL solution was measured for each of the control and the Examples 4 to 7. The result is shown in Table 3.

TABLE 2

pH

| | 0 minute | 3 minutes |
|---|---|---|
| Control | 6.2 | 5.3 |
| Example 4 | 6.6 | 6.6 |
| Example 5 | 6.3 | 6.2 |
| Example 6 | 6.2 | 6.0 |
| Example 7 | 6.5 | 6.2 |

TABLE 3

| | pH |
|---|---|
| Control | 3.1 |
| Example 4 | 3.3 |
| Example 5 | 3.2 |
| Example 6 | 3.1 |
| Example 7 | 3.2 |

As shown in the results on Tables 2 and 3, when Capparis masaikai Levl. is compounded, it was possible to make the pH value rise without declining the pH (equal to or less than about 5.5) which damages on the tooth as seen in the case of the control.

It is thought that this effect is not merely neutralization effect by adding Capparis masaikai Levl. (See Table 3) but this is the result because the saliva promoted secretion by adding Capparis masaikai Levl. buffered the pH decline by glucose and citric acid.

Example of formula for the food composition and the oral composition concerning the present invention is shown as follows:

| (Combination example 1; Chewing gum) | | |
|---|---|---|
| Gum base: | 100 | part by weight |
| Citric acid: | 1 | part by weight |
| Perfume: | 1 | part by weight |
| 50% of ethanol extractive of Capparis masaikai | 1 | part by weight |
| Levl.: | | |
| (Combination example 2; Juice) | | |
| Concentrated fruit juice: | 5.0 | part by weight |
| Fructose, glucose, liquid sugar: | 10.0 | part by weight |
| L-ascorbic acid: | 0.2 | part by weight |
| Water: | 85.0 | part by weight |
| Perfume: | 0.1 | part by weight |
| 50% of ethanol extractive of Capparis masaikai | 0.5 | part by weight |
| Levl.: | | |
| (Combination example 3; Candy) | | |
| Sugar: | 120.0 | part by weight |
| Starch syrup: | 100.0 | part by weight |
| Citric acid: | 4.0 | part by weight |
| Perfume: | 0.4 | part by weight |
| 50% of ethanol extractive of Capparis pterocarpa | 1.0 | part by weight |
| Chun: | | |
| (Combination example 4; Cracker) | | |
| Hard flour: | 100.0 | part by weight |
| Weak flour: | 100.0 | part by weight |
| White superior soft sugar: | 30.0 | part by weight |
| Powder sugar: | 40.0 | part by weight |
| Shortening: | 100.0 | part by weight |
| Sodium bicarbonate: | 0.6 | part by weight |
| Whole milk powder: | 4.0 | part by weight |
| Water: | 20.0 | part by weight |
| 50% of ethanol extractive of Centella: | 1.0 | part by weight |

### Effect of the Invention

The sialagogue, and food composition and oral composition containing thereof, concerning the present invention, raise the self-cleansing action in the mouth by promoting salivary secretion having more than fixed quantity consecutively for a long time, and cure and/or prevent various disorder, which is caused by decline of amount of salivary secretion, such as dental caries.

What is claimed is:

1. Sialagogue wherein one or more kind of at least plant or extract thereof, selected from the group of the plant belonging to Capparidaceae and the plant belonging to Umbelliferae are contained.

2. The Sialagogue wherein one or more kind of at least plant or extract thereof, selected from the group of the plant belonging to Capparidaceae and Capparis, and the plant belonging to Umbelliferae and Centella are contained.

3. The sialagogue wherein one or more kind of at least plant or extract thereof, selected from the group of Capparis pterocarpa Chun, Capparis masaikai Levl. and Capparis asiatica (L.) Urban are contained.

4. The sialagogue claimed in claim 1, wherein organic acid is contained.

5. The sialagogue claimed in claim 2, wherein organic acid is contained.

6. The sialagogue claimed in claim 3, wherein organic acid is contained.

7. The sialagogue claimed in claim 1, wherein organic acid and sugar alcohol are contained.

8. The sialagogue claimed in claim 2, wherein organic acid and sugar alcohol are contained.

9. The sialagogue claimed in claim 3, wherein organic acid and sugar alcohol are contained.

10. The sialagogue claimed in claim 1, wherein organic acid, sugar alcohol and at least one selected from the group consisting of peppermint oil, cinnamon oil, menthol and cinnamic aldehyde are contained.

11. The sialagogue claimed in claim 2, wherein organic acid, sugar alcohol and at least one selected from the group consisting of peppermint oil, cinnamon oil, menthol and cinnamic aldehyde are contained.

12. The sialagogue claimed in claim 3, wherein organic acid, sugar alcohol and at least one selected from the group consisting of peppermint oil, cinnamon oil, menthol and cinnamic aldehyde are contained.

13. The sialagogue claimed in claim 1, wherein fermentable sugar is contained.

14. The sialagogue claimed in claim 2, wherein fermentable sugar is contained.

15. The sialagogue claimed in claim 3, wherein fermentable sugar is contained.

16. The sialagogue claimed in claim 1, wherein fermentable sugar and sugar alcohol are contained.

17. The sialagogue claimed in claim 2, wherein fermentable sugar and sugar alcohol are contained.

18. The sialagogue claimed in claim 3, containing fermentable sugar and sugar alcohol.

19. The sialagogue claimed in claim 1, wherein fermentable sugar, sugar alcohol and at least one selected from the group consisting of peppermint oil, cinnamon oil, menthol and cinnamic aldehyde are contained.

20. The sialagogue claimed in claim 2, wherein fermentable sugar, sugar alcohol and at least one selected from the group consisting of peppermint oil, cinnamon oil, menthol and cinnamic aldehyde are contained.

21. The sialagogue claimed in claim 3, wherein fermentable sugar, sugar alcohol and at least one of selected from the group consisting peppermint oil, cinnamon oil, menthol and cinnamic aldehyde are contained.

22. The sialagogue claimed in claim 1, wherein one or more kind of which selected from the group of organic acid, fermentable sugar, sugar alcohol, peppermint oil, cinnamon oil, menthol and cinnamic aldehyde are contained.

23. The sialagogue claimed in claim 2, wherein one or more kind of which selected from the group of organic acid, fermentable sugar, sugar alcohol, peppermint oil, cinnamon oil, menthol and cinnamic aldehyde are contained.

24. The sialagogue claimed in claim 3, wherein one or more kind of which selected from the group of organic acid, fermentable sugar, sugar alcohol, peppermint oil, cinnamon oil, menthol and cinnamic aldehyde are contained.

25. The food composition wherein sialagogue claimed in claim 1 is compounded.

26. The food composition wherein sialagogue claimed in claim 2 is compounded.

27. The food composition wherein sialagogue claimed in claim 3 is compounded.

28. The food composition wherein sialagogue claimed in claim 4 is compounded.

29. The food composition wherein sialagogue claimed in claim 5 is compounded.

30. The food composition wherein sialagogue claimed in claim 6 is compounded.

31. The food composition wherein sialagogue claimed in claim 7 is compounded.

32. The food composition wherein sialagogue claimed in claim 8 is compounded.

33. The food composition wherein sialagogue claimed in claim 9 is compounded.

34. The food composition wherein sialagogue claimed in claim 10 is compounded.

35. The food composition wherein sialagogue claimed in claim 11 is compounded.

36. The food composition wherein sialagogue claimed in claim 12 is compounded.

37. The food composition wherein sialagogue claimed in claim 13 is compounded.

38. The food composition wherein sialagogue claimed in claim 14 is compounded.

39. The food composition wherein sialagogue claimed in claim 15 is compounded.

40. The food composition wherein sialagogue claimed in claim 16 is compounded.

41. The food composition wherein sialagogue claimed in claim 17 is compounded.

42. The food composition wherein sialagogue claimed in claim 18 is compounded.

43. The food composition wherein sialagogue claimed in claim 19 is compounded.

44. The food composition wherein sialagogue claimed in claim 20 is compounded.

45. The food composition wherein sialagogue claimed in claim 21 is compounded.

46. The food composition wherein sialagogue claimed in claim 22 is compounded.

47. The food composition wherein sialagogue claimed in claim 23 is compounded.

48. The food composition wherein sialagogue claimed in claim 24 is compounded.

49. The oral composition wherein sialagogue claimed in claim 1 is compounded.

50. The oral composition wherein sialagogue claimed in claim 2 is compounded.

51. The oral composition wherein sialagogue claimed in claim 3 is compounded.

52. The oral composition wherein sialagogue claimed in claim 4 is compounded.

53. The oral composition wherein sialagogue claimed in claim 5 is compounded.

54. The oral composition wherein sialagogue claimed in claim 6 is compounded.

55. The oral composition wherein sialagogue claimed in claim 7 is compounded.

56. The oral composition wherein sialagogue claimed in claim 8 is compounded.

57. The oral composition wherein sialagogue claimed in claim 9 is compounded.

58. The oral composition wherein sialagogue claimed in claim 10 is compounded.

59. The oral composition wherein sialagogue claimed in claim 11 is compounded.

60. The oral composition wherein sialagogue claimed in claim 12 is compounded.

61. The oral composition wherein sialagogue claimed in claim 13 is compounded.

62. The oral composition wherein sialagogue claimed in claim 14 is compounded.

63. The oral composition wherein sialagogue claimed in claim 15 is compounded.

64. The oral composition wherein sialagogue claimed in claim 16 is compounded.

65. The oral composition wherein sialagogue claimed in claim 17 is compounded.

66. The oral composition wherein sialagogue claimed in claim 18 is compounded.

67. The oral composition wherein sialagogue claimed in claim 19 is compounded.

68. The oral composition wherein sialagogue claimed in claim 20 is compounded.

69. The oral composition wherein sialagogue claimed in claim 21 is compounded.

70. The oral composition wherein sialagogue claimed in claim 22 is compounded.

71. The oral composition wherein sialagogue claimed in claim 23 is compounded.

72. The oral composition wherein sialagogue claimed in claim 24 is compounded.
